(19) United States
(12) Patent Application Publication
(10) Pub. No.: US 2022/0016282 A1
(43) Pub. Date: Jan. 20, 2022

(54) Self-Disinfection Device

(71) Applicant: Gerald Ho Kim, Ontario, CA (US)

(72) Inventor: Gerald Ho Kim, Ontario, CA (US)

(21) Appl. No.: 17/374,121

(22) Filed: Jul. 13, 2021

Related U.S. Application Data
(60) Provisional application No. 63/051,887, filed on Jul. 14, 2020.

(51) Int. Cl. A61L 2/10; G06F 1/16

(52) U.S. Cl. CPC A61L 2/10; G06F 1/1626; A61L 2202/16; A61L 2202/11; G06F 1/1637

(57) ABSTRACT

A self-disinfection device includes an ultraviolet C (UVC) light-emitting diode (LED) illuminator, which includes a UVC LED chip and a screen. The UVC LED chip that is mounted on a UVC LED package or a heat spreader, which is mounted to a frame of an electronic device. A UVC light emitted by the UVC LED chip is coupled into the screen as a waveguide structure and the UVC light is extracted by a coating or organic medium.

### CROSS REFERENCE TO RELATED PATENT APPLICATION(S)

The present disclosure claims the priority benefit of U.S. Patent Application No. 63/051,887, filed 14 Jul. 2020, the content of which being incorporated by reference in its entirety.

### TECHNICAL FIELD

The present disclosure generally relates to disinfection devices and, more particularly, to a self-disinfection device.

### BACKGROUND

Unless otherwise indicated herein, the approaches described in this section are not prior art to the claims in this application and are not admitted as prior art by inclusion in this section.

The increasing germicidal contamination and cross-contamination from using portable electronic devices such as mobile phones, tablet, game consoles, touch-screens, bank tele-machines, and laptops has been a concern for public safety and wide-spread germs prevention. Many disinfection methods such as chemical, heat and ultraviolet (UV) light disinfection have been developed in the past. This invention uses ultraviolet C (UVC) light-emitting diode (LED) light that is integrated into electronic devices to illuminate the surface of the electronic device so that it can disinfect itself. For example, a typical UVC disinfection system works by putting electronic devices in a confined box where the UVC light illuminates the surface of the electronic device. In this case the electronic device has to be placed inside the UVC illuminating box and the disinfection device has to be carried along with the portable device.

### SUMMARY

The following summary is illustrative only and is not intended to be limiting in any way. That is, the following summary is provided to introduce concepts relating to a heat sink for thermal management in an electronic apparatus. Select embodiments of the novel and non-obvious technique are further described below in the detailed description. Thus, the following summary is not intended to identify essential features of the claimed subject matter, nor is it intended for use in determining the scope of the claimed subject matter.

The present disclosure focuses on the portable device to self-disinfected as it is needed. The UVC LED will be placed inside of the electronic device and it will transmit the UVC light through a UVC transmitting glass or plate.

### BRIEF DESCRIPTION OF THE DRAWINGS

The accompanying drawings are included to provide a further understanding of the disclosure and are incorporated in and constitute a part of the present disclosure. The drawings illustrate embodiments of the disclosure and, together with the description, serve to explain the principles of the disclosure. It is appreciable that the drawings are not necessarily in scale as some components may be shown to be out of proportion than the size in actual implementation in order to clearly illustrate the concept of the present disclosure.

FIG. 1 shows a basic concept of UVC LED illuminator with UVC LEDs in accordance with the present disclosure.

FIG. 2 show an example of UVC LED packages illuminating UVC light onto a surface of a screen in accordance with the present disclosure.

FIG. 3 shows an example implementation of a self-disinfection device in accordance with the present disclosure.

FIG. 4 shows an example implementation of a self-disinfection device in accordance with the present disclosure.

FIG. 5 shows an example implementation of a self-disinfection device in accordance with the present disclosure.

FIG. 6 shows an example implementation of a self-disinfection device in accordance with the present disclosure.

FIG. 7 shows an example implementation of a self-disinfection device in accordance with the present disclosure.

### DETAILED DESCRIPTION OF PREFERRED EMBODIMENTS

### Overview

Most portable electronic devices in hospitals or public areas have no protection against any germs or viruses. The electronic device can be portable or stationary such as a bank tele-machine where a touch screen is present. The touchscreen has to be constantly disinfected so that it will not transmit any germs or viruses. UVC light (200 nm to 280 nm) can shine onto the surface of the portable electronic devices to disinfect. In this case, the electronic device has to be put into the disinfection chamber or be exposed with UVC light. The self-disinfection concept of uniformly illuminating all touching area or glass area is very difficult to implement since UVC light can only transmit through a few types of materials such as UV quartz, sapphire, fluorocarbon polymers, and others. All the touchscreen surface has to be uniformly illuminated with UVC light in order to disinfect the surface properly.

The UVC light is illuminated inside of the window or touchscreen and a special coating is applied to allow the UVC light to uniformly leak throughout the window area. The window or touchscreen that transmit the UVC light will trap or waveguide the UVC light throughout the screen of the electronic device and illuminate the UVC light uniformly. The portable electronic device can illuminate the UVC light frequently or on-demanded as user activates the disinfection feature. The disinfection time and strength can be programed on the electronic device to meet battery or electrical requirements.

The major application of this invention is developing a self-disinfecting mobile phone device where the UVC LED is imbedded inside of the mobile phone. The UVC LED is powered by the battery and UVC light is injected into the touchscreen of the mobile phone when user activate the disinfection function or it turns on from auto programming. The UVC light propagates through the inside of the touchscreen and is uniformly emitted from the surface of the touchscreen. A special coating is applied on the touchscreen to help uniformly emit the UVC light, since only a small amount of UVC light typically escapes from the touchscreen and this UVC light may not uniformly illuminate the whole touchscreen area, but where the special coating is applied it can help to uniformly emit UVC light from the touchscreen.

The special coating is applied to the window or touchscreen to bring the light our from the window or touchscreen. Depending on the application, it is not necessary to apply the special coating and trap the UVC light inside of the window or touchscreen. For instance, the UVC light can be harmful with prolonged eyes and skin exposure, therefore the UVC light will be only emitted when germs or viruses are present at the surface of the window or touchscreen. If no special coating is applied on the surface of the window or touchscreen, a lot of UVC light will be trapped inside of the window or touchscreen due to a high internal reflection of the air-glass boundary. In the case of no special coating applied to the surface of the window, the UVC light can be emitted by any water droplets or fingerprints on the screen caused by airborne saliva or a contaminated hand.

The self-disinfection device contains a window or touchscreen for electronics devices where the UVC light can be trapped or contained inside of the screen. In order to disinfect the device, the UVC light has to be emitted from the screen and effectively penetrate a contaminated substance such as a droplet, spit, fingerprint, moisture or any organic substance on the surface of the screen. However, the UVC light will emit more effectively where these materials are touching the screen, due to a better UVC light transmission at the contacted area. To create a uniform illumination of the screen area a structure to scatter the UVC light inside of the screen is embedded by making channels and grooves by laser engraving or a dicing saw. The structure gives a scatter point inside of the screen and the UVC light is scattered at the structure and comes out of the screen. The amount of scattering UVC light can be controlled by the location and depth of the structure embedded on the surface or inside of the screen. In this case the structure can be patterned on the screen to emit a uniform illumination pattern.

The uniform illumination on the screen can be designed by both a special coating or the scattering structure on the surface of the window or touchscreen. The special coating can be applied on the emission face as an anti-refection or partial reflection coating. The special coating can be applied as a gradient pattern to control the uniformity of the UVC light emitted from the screen. The same effect can be created by the scattering structure to control the uniform illumination of the screen by designing a proper pattern of the scattering structure and controlling the depth of the scattering structure. The scattering pattern of the structure is unique to match with the illumination pattern of the UVC LEDs making a uniform illumination throughout the whole screen area. The unique scatter patterns are not limited to straight line in groove or channel and it can consist of a curve, square or circular elements where it can create a uniform illumination pattern from the screen.

### Illustrative Implementations

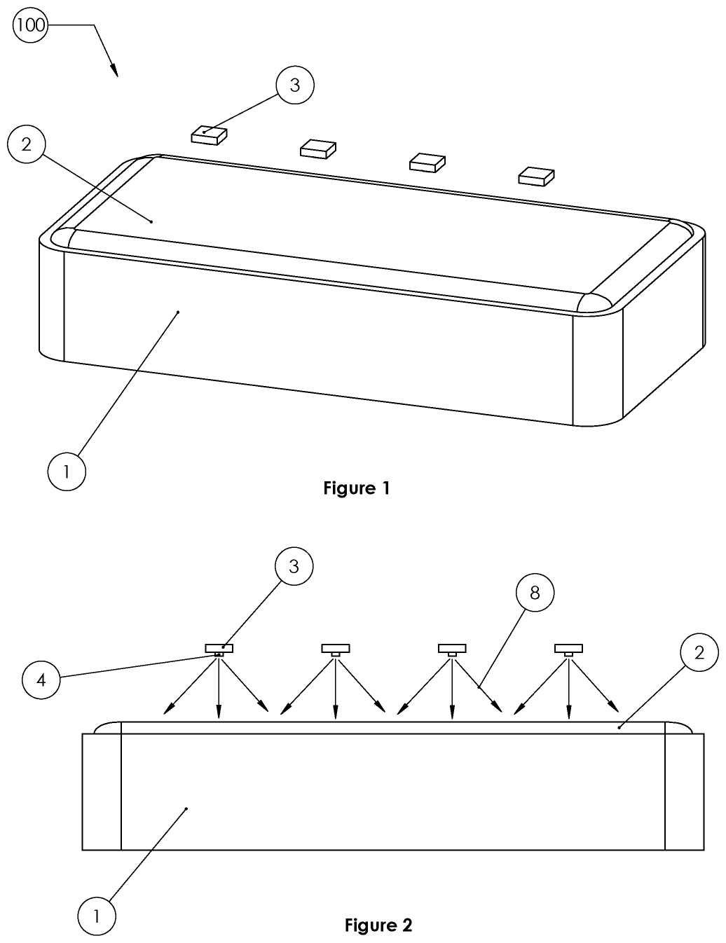

FIG. 1 shows a basic concept of UVC LED illuminator (100) with UVC LEDs. An electronic device (1) is placed under UVC LED packages (3) and a screen (2) is directly underneath of the UVC LED packages (3).

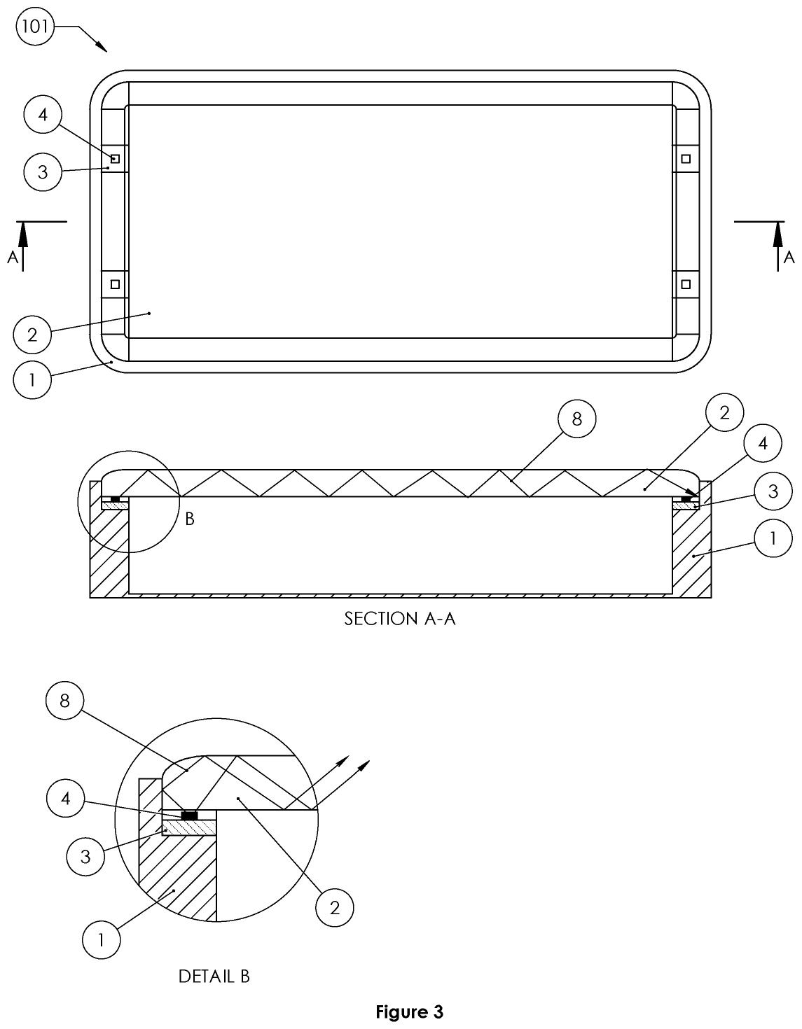

FIG. 2 show an illustration of the UVC LED packages (3) illuminate UVC light (8) onto the surface of the screen (2). The UVC LED packages (3) contains UVC LED chips (4) that emit UVC light (8) to illuminate the screen (2) in the electronic device (1).

FIG. 3 shows a self-disinfection device (101) where it contains the UVC LED packages (3) and its UVC LED chips (4) attached to the frame of the electronic device (1). The UVC light (8) is emitted from the UVC LED packages (3) and enters to the screen (2) and the UVC light is trapped as waveguide due to a total internal refection. The UVC LEDs are placed underneath of the screen (2) and integrated with the screen (2). Also, the UVC LED packages (3) is directly mounted on the frame of the electronic device (1) for thermal dissipation. The detail view ‘B’ shows the edge of the screen (2) is curved to capture all UVC light (8) to be trapped or contains inside of the screen (2) so that the light can be emitted uniformly throughout the whole area of the screen (2).

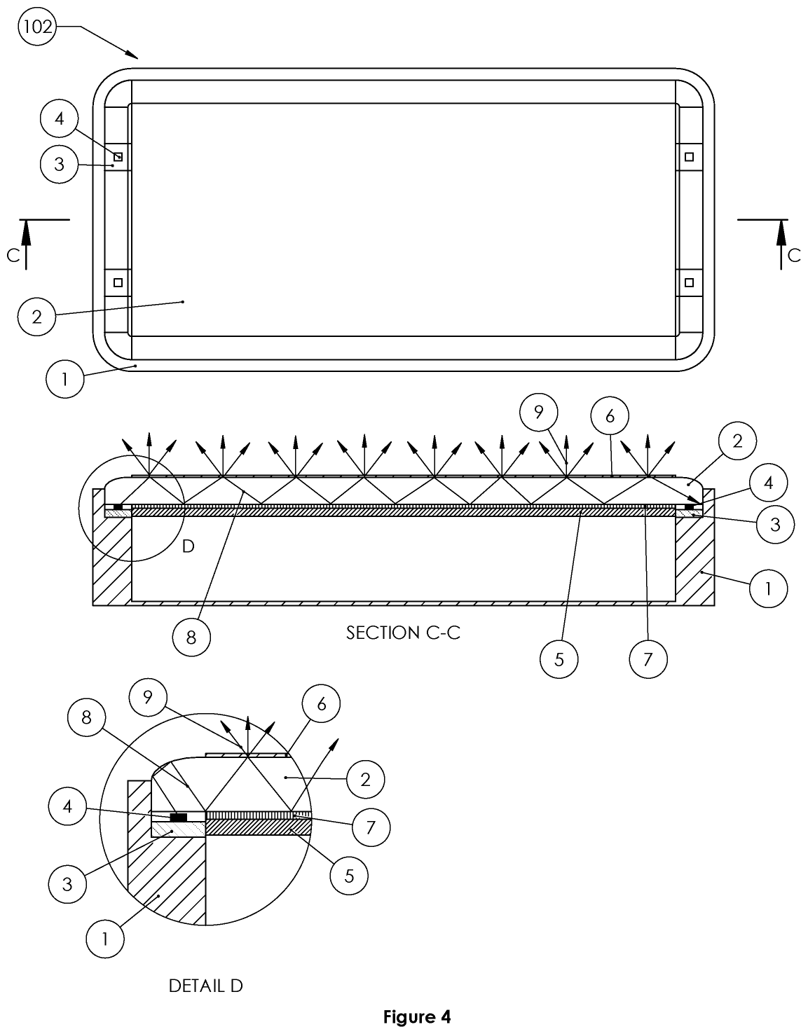

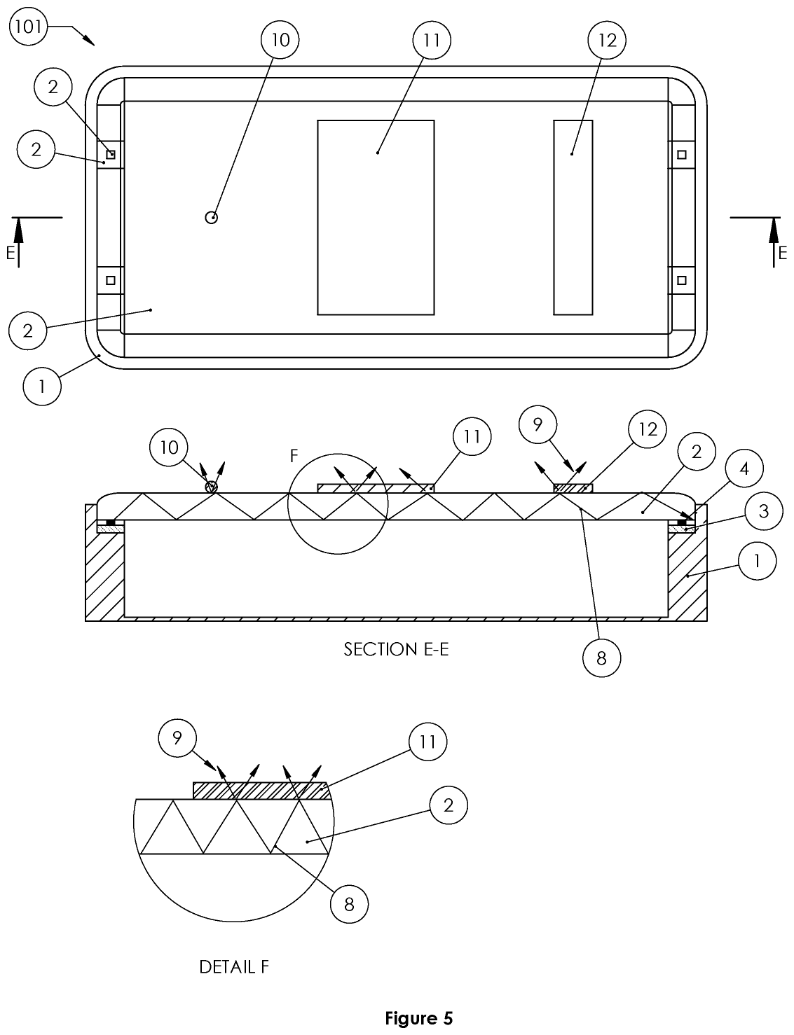

FIG. 4 shows a self-disinfection device (102) similar to the self-disinfection device (101). The self-infection device (102) contains the UVC LED packages (3) and its UVC LED chips (4) attached to the frame of the electronic device (1). The UVC light (8) is emitted from the UVC LED packages (3) and enter to the screen (2) and the UVC light is trapped as waveguide due to a total internal refection. However, the self-disinfection device (102) added a special coating (6) to bring out the UVC light (8) from the screen (2) and also a high reflection coating (7) and display element (5) is added to the opposite side of the special coating (6). Due to the special coating (6) the UVC light (8) is escaped from the screen (2) and propagate to the escaped UVC light (9) for disinfecting all germs and virus.

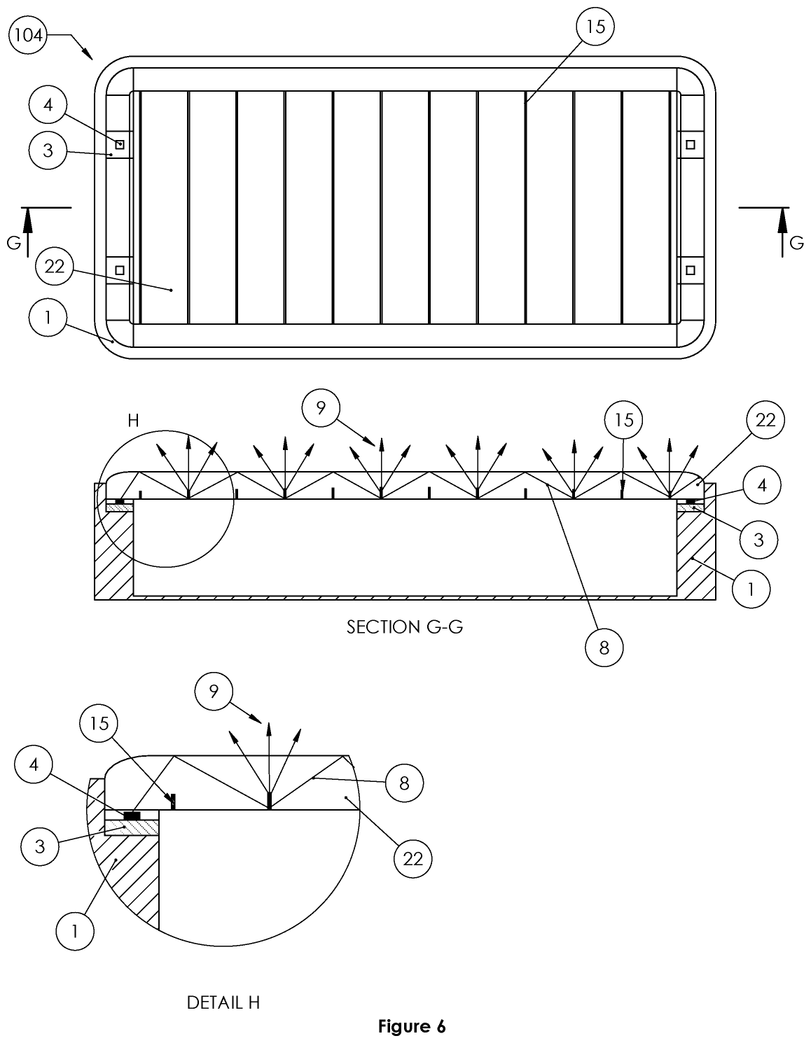

FIG. 5 shows the self-disinfection device (101) where a droplet (10), organic stain (11) or fingerprint (12) are contaminated on the surface of the screen (2). It demonstrates how the UVC light (8) escapes from the inside of the screen (8) by the droplet (10), organic stain (11) or fingerprint (12). These droplet (10), organic stain (11) or fingerprint (12) can be disinfected as the UVC light (8) is transmitted through these mediums and propagate as escaped UVC light (9).

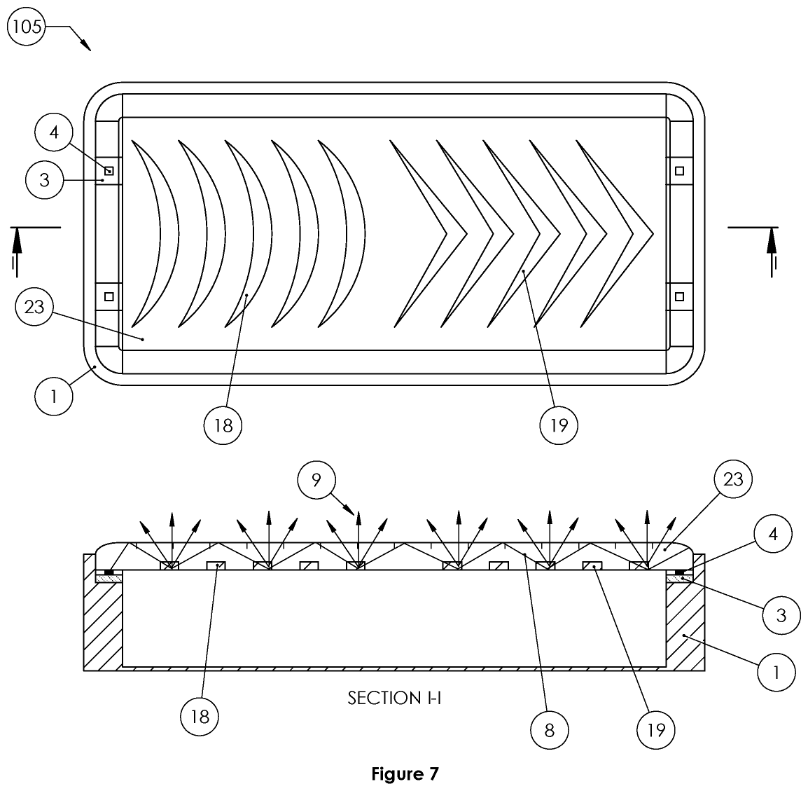

FIG. 6 shows a self-disinfection device (104) where the screen (22) is modified to scatter the UVC light (8) by structures (15) to illuminate uniformly. The self-disinfection device (104) contains the UVC LED packages (3) and its UVC LED chips (4) attached to the frame of the electronic device (1). The UVC light (8) is emitted from the UVC LED packages (3) and enters to the screen (22) and the UVC light is trapped by a waveguide due to a total internal refection. The screen (22) contains multiple structures (15) to bring the UVC light (8) out of the screen (22) and the scattered UVC light (9) for disinfecting all germs and viruses on the surface of the screen (22).

FIG. 7 shows a self-disinfection device (105) where the screen (23) has scattering structures (18, 19). The structures (18, 19) that scatters the UVC light (8) can be made with any patterns to create the uniform illumination in the self-disinfection device (105). The self-disinfection device (105) where it contains the UVC LED packages (3) and its UVC LED chips (4) attached to the frame of the electronic device (1). The UVC light (8) is emitted from the UVC LED packages (3) and enters the screen (23) and the UVC light (8) is trapped by a waveguide due to a total internal refection. The screen (23) contains multiple structures (18, 19) to bring the UVC light out of the screen (23) and the UVC light (8) can disinfect all germs and viruses on the surface of the screen (23) by the escaped UVC light (9).

### Highlight of Select Features

In view of the above, select features in accordance with the present disclosure are highlighted below.

In one aspect, a self-disinfection device may include a UVC LED illuminator. The UVC LED illuminator may include a UVC LED chip and a screen. The UVC LED chip may be mounted on a UVC LED package and the UVC LED package may be mounted to a frame of an electronic device. A UVC light emitted by the UVC LED chip may be coupled into the screen as a waveguide structure and the UVC light may be extracted by a coating or organic medium.

In some implementations, the screen may be made with UVC transmitting sapphire glass.

In some implementations, the screen may be made with UVC transmitting quartz glass.

In some implementations, the screen may be made with UVC transmitting polymers.

In some implementations, the electronic device may include a mobile phone.

In some implementations, the electronic device may include a portable computer.

In some implementations, the electronic device may include touchscreen monitor.

In some implementations, the coating may be anti-reflection or partial anti-reflection coating.

In some implementations, the organic medium may be biological contaminated droplet.

In some implementations, the organic medium may include a fingerprint.

In another aspect, a self-disinfection device may include a UVC LED illuminator. The UVC LED illuminator may include a UVC LED chip and a screen. The UVC LED chip may be mounted on a heat spreader and the heat spreader may be mounted to a frame of an electronic device. A UVC light emitted by the UVC LED chip may be coupled into the screen as a waveguide structure and the UVC light may be extracted by a structure that is imbedded in the screen.

In some implementations, the screen may be made with UVC transmitting sapphire glass.

In some implementations, the screen may be made with UVC transmitting quartz glass.

In some implementations, the screen may be made with UVC transmitting polymers.

In some implementations, the structure may include a laser engraved channel.

In some implementations, the channel depth may be varied to emit uniform illumination.

In some implementations, the groove depth may be varied to emit uniform illumination.

In some implementations, the structure may include a dicing saw groove.

In some implementations, the structure may be patterned to make a uniform screen illumination.

### ADDITIONAL NOTES AND CONCLUSION

The herein-described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Further, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A self-disinfection device, comprising:
an ultraviolet C (UVC) light-emitting diode (LED) illuminator comprising:
a UVC LED chip that is mounted on a UVC LED package and the UVC LED package is mounted to a frame of an electronic device; and
a screen where a UVC light emitted by the UVC LED chip is coupled into the screen as a waveguide structure and the UVC light is extracted by a coating or organic medium.

2. The self-disinfection device of claim 1, wherein the screen is made with UVC transmitting sapphire glass.

3. The self-disinfection device of claim 1, wherein the screen is made with UVC transmitting quartz glass.

4. The self-disinfection device of claim 1, wherein the screen is made with UVC transmitting polymers.

5. The self-disinfection device of claim 1, wherein the electronic device is mobile phone.

6. The self-disinfection device of claim 1, wherein the electronic device is portable computer.

7. The self-disinfection device of claim 1, wherein the electronic device is touchscreen monitor.

8. The self-disinfection device of claim 1, wherein the coating is anti-reflection or partial anti-reflection coating.

9. The self-disinfection device of claim 1, wherein the organic medium is biological contaminated droplet.

10. The self-disinfection device of claim 1, wherein the organic medium is fingerprint.

11. A self-disinfection device, comprising:
an ultraviolet C (UVC) light-emitting diode (LED) illuminator comprising:
a UVC LED chip that is mounted on a heat spreader and the heat spreader is mounted to a frame of an electronic device; and
a screen where a UVC light emitted by the UVC LED chip is coupled into the screen as a waveguide structure and the UVC light is extracted by a structure that is imbedded in the screen.

12. The self-disinfection device of claim 11, wherein the screen is made with UVC transmitting sapphire glass.

13. The self-disinfection device of claim 11, wherein the screen is made with UVC transmitting quartz glass.

14. The self-disinfection device of claim 11, wherein the screen is made with UVC transmitting polymers.

15. The self-disinfection device of claim 11, wherein the structure is laser engraved channel.

16. The self-disinfection device of claim 15, wherein the channel depth is varied to emit uniform illumination.

17. The self-disinfection device of claim 16, wherein the groove depth is varied to emit uniform illumination.

18. The self-disinfection device of claim 11, wherein the structure is dicing saw groove.

19. The self-disinfection device of claim 11, wherein the structure is patterned to make a uniform screen illumination.
